(19) United States
(12) Patent Application Publication
(10) Pub. No.: US 2005/0164242 A1
(43) Pub. Date: Jul. 28, 2005

(54) Method for minimizing contamination of crops and products derived therefrom

(75) Inventors: Peter J. Shortridge, St. Paul, MN (US); Kate Leavitt Thoresen, Eagan, MN (US); Michelle A. Novak, Lakeville, MN (US); Amy A. Nankivil, Winona, MN (US); Heather Ninow, St. Paul, MN (US); Kelly Shea Dougherty, Colorado Springs, CO (US)

Correspondence Address: FISH & RICHARDSON P.C., PO BOX 1022, MINNEAPOLIS, MN, 55440-1022, US

(73) Assignee: Northland Seed & Grain Corp., a Minnesota corporation

(21) Appl. No.: 10/966,830

(22) Filed: Oct. 15, 2004

Related U.S. Application Data
(63) Continuation of application No. 09/641,114, filed on Aug. 17, 2000, now abandoned.
Application No. 09/641,114 is a continuation-in-part of application No. 09/251,953, filed on Feb. 17, 1999, now abandoned.

(51) Int. Cl. C12Q 1/68; A01H 5/00; G06F 17/60

(52) U.S. Cl. 435/6; 705/1

(57) ABSTRACT

A method of minimizing contamination of processed grain and processed food products is disclosed. The method can include selecting seeds which are known to contain a desired variety, inspecting a grower's operation and machinery to certify that the operation is free of contamination prior to harvest, harvesting the crop, inspecting the processing facility to verify that its operation is free of contamination prior to harvest and processing the crop into processed grain or food products. The method is suitable for minimizing or preventing contamination of non-GMO processed grain by GMO seeds, or for minimizing or preventing contamination of GMO processed grain by non-GMO seeds.

### TECHNICAL FIELD

This invention relates in general to a method of preparing processed grains and processed food products. More particularly the invention relates to a method of preparing processed grain that has little or no contamination by unwanted types of grain, such as contamination of non-genetically modified processed grain by genetically-modified seeds.

### BACKGROUND

It can be seen that there is a need for a method of creating and preserving the identity of non-genetically modified (non-GMO) seeds and grains. Over the past number of years, genetically modified and genetically engineered (GMO) seeds and grains are becoming common place within the agriculture industry. The prevalence of these genetically altered products has given rise to a market for non-genetically modified seeds, grains, and processed products created therefrom. Similarly, the prevalence of different types of genetically modified grain has created a need for a method to ensure that such types of grain are not contaminated by non-genetically modified grains.

It can also be seen that there is a need for a method to ensure that processed grain and processed food products are not contaminated by unwanted seeds or seed material during the process of bringing these products to market.

To date, no method of growing, harvesting, processing, packaging, and distributing non-genetically modified seeds and grains utilizes a comprehensive process which encompasses the entire food production system from planting a crop to a finished product. Unfortunately, the production of non-genetically modified processed grain, and processed food products, requires such a comprehensive process if the consumer is to have confidence that food products made from non-genetically modified seeds, grains, and processed products have not been contaminated somewhere in the production process.

### SUMMARY

To overcome the limitations in the prior art described above, and to overcome other limitations that will become apparent upon reading and understanding the present specification, the present invention discloses a method of creating and preserving the identity of non-genetically modified processed grains, and processed grain products.

The present invention provides a method of preparing non-GMO processed grains, and processed food products, such that contamination by GMO seeds is minimized or even eliminated. The method can include selecting seeds which are known to contain non-GMO varieties, planting the non-GMO crop, inspecting grower's operation and machinery to certify that the operation is free of contamination prior to harvest, harvesting the crop, inspecting the processing facility to verify that its operation is free of contamination prior to harvest, tracking all containers holding non-GMO crops each time the crop is moved, and processing the non-GMO crops into containers for shipment where the containers possess tracking information.

This methodology is readily adaptable to the segregation of genetically modified crops such that processed grain and food products can be prepared therefrom with little or no contamination by non-GMO seeds or crop material. Processed food ingredients and processed food products made from such processed grain can also be prepared utilizing the methodology. The methodology can also be employed to provide certification of purity for a GMO or non-GMO crop, or to provide certification of purity for a GMO or non-GMO processed food product.

The invention features a method for preparing a non-genetically modified processed food product. This method comprises certifying that a harvested crop was processed under conditions effective for producing a processed food product that contains 5% or less genetically modified crop material. The processing conditions can be effective for producing a processed food product containing 0.01% or less genetically modified crop material. Certifying the processing conditions can comprise obtaining genetic test results or application susceptibility test results indicating that the processed food product contains 0.01% or less genetically modified crop material. Certifying the processing conditions can also comprise inspecting for contamination by genetically modified seeds, prior to the harvesting step, one or more storage bins for the crop, and inspecting for contamination by genetically modified seeds, prior to the processing step, one or more processing plants that are to process the crop. Certifying the processing conditions can also comprise establishing a lot identification number for the crop prior to the processing step and tracking the lot identification number during the processing step. The lot identification number can be established when the crop is harvested.

The method can also comprise certifying that non-genetically modified seeds were grown under conditions effective for harvesting the crop such that the crop contains 5% or less genetically modified seeds after harvest. The conditions for harvest can be effective for producing a crop containing 0.01% or less genetically modified seeds and may comprise obtaining genetic test results or application susceptibility test results indicating that the non-genetically modified seeds contain 0.01% or less genetically modified seeds. In some embodiments, certifying a harvested crop can comprise testing non-genetically modified seeds to be used for planting for contamination by genetically modified seeds, such a test carried out prior to planting. Certifying a harvested crop can also comprise inspecting the crop for contamination by genetically modified plants prior to harvesting the crop.

Non-genetically modified seeds used in the method can be seeds of a large-seeded grain crop, e.g., corn. Non-genetically modified processed food products that can be made from corn by the method include corn sweetener, corn gluten meal, corn starch, corn meal and corn flour. Non-genetically modified seeds can also be soybean seeds, in which case non-genetically modified processed food products that can be made include soy lecithin, soy flour, soy sauce, soy milk, soy desserts, textured soy protein, tofu and soy meal.

The invention also features a method for minimizing contamination of genetically modified processed grain, genetically modified processed food products, or pharmaceuticals and nutrients made from a genetically modified crop. Such a method can comprise certifying that a harvested genetically modified crop contains 5% or less non-genetically modified seeds and certifying that the crop was processed under conditions effective for producing genetically modified processed grain containing 5% or less non-genetically modified seeds.

The crop can be certified at harvest to contain 0.01% or less non-genetically modified seeds. The crop can be certified at harvest by obtaining genetic test results or application susceptibility test results indicating that the genetically modified seeds contain 0.01% or less non-genetically modified seeds. The method can also include testing genetically modified seeds to be used for growing the crop for contamination by non-genetically modified seeds prior to planting the genetically modified seeds, inspecting the crop for contamination by non-genetically modified plants prior to harvesting the crop, or verifying that equipment used to grow and harvest the crop was cleaned-down prior to harvest.

The processing conditions in the method can be effective for producing processed grain containing 0.01% or less non-genetically modified seeds. Certifying the processing conditions can include obtaining genetic test results or application susceptibility test results indicating that the processed grain contains 0.01% or less non-genetically modified seeds. In some embodiments, certifying the processing conditions can include inspecting for contamination by non-genetically modified seeds, prior to harvesting the crop, one or more storage bins that are to store the crop and inspecting for contamination by non-genetically modified seeds, prior to processing the crop, one or more processing plants that are to process the crop. A lot identification number can be established for the crop prior to the processing step, e.g, when the crop is harvested.

The invention also features a method for producing a harvested crop, comprising testing non-genetically modified seeds for contamination by genetically modified seeds prior to planting and growing the non-genetically modified seeds, testing a crop harvested from the non-genetically modified seeds for contamination by genetically modified seeds, and certifying that the harvested crop contains 5% or less genetically modified seeds, based on the testing. The certification can be based on inspecting the crop for contamination by genetically modified plants while the crop is growing, or on inspecting for contamination by genetically modified seeds one or more storage bins for the crop, prior to harvesting the crop. The certification can also be based on inspecting, prior to harvest, equipment used to grow and harvest the crop. Testing prior to planting can include obtaining genetic test results or application susceptibility test results indicating that the non-genetically modified seeds contain 0.01% or less genetically modified seeds. Testing a harvested crop can include obtaining genetic test results indicating that the harvested crop contains 0.01% or less genetically modified seeds.

The invention also features a method for minimizing contamination of a non-genetically modified processed food product. The method includes inspecting for contamination by genetically modified crop material a processing facility that is to process a harvested crop, prior to processing the harvested crop. The method can also include testing the harvested crop for contamination by genetically modified crop material prior to processing of the harvested crop by the processing facility to make the non-genetically modified processed food product. The method can also include certifying that the processed food product contains 5% or less genetically modified crop material after processing the harvested crop, based on the inspecting and testing. Testing can include obtaining genetic test results indicating that the processed food product contains 0.01% or less genetically modified crop material. Certifying can include establishing a lot identification number for the crop prior to the testing step and may involve testing a plurality of samples obtained during processing of the crop.

These and various other advantages and features of novelty which characterize the invention are pointed out with particularity in the claims annexed hereto and form a part hereof. However, for a better understanding of the invention, its advantages, and the objects obtained by its use, reference can be made to the drawings which form a further part hereof, and to accompanying descriptive matter, in which there are illustrated and described specific examples of the invention.

### DESCRIPTION OF DRAWINGS

Referring now to the drawings in which like reference numbers represent corresponding parts throughout:

FIG. 1 is a process flow chart for a method of creating and preserving the identity of non-genetically modified seeds and grains according to one example embodiment of the present invention for steps prior to planting through crop harvest.

FIG. 2 is a process flow chart for a method of creating and preserving the identity of non-genetically modified seeds and grains according to one example embodiment of the present invention for steps from crop harvest through delivery of products to customers.

FIG. 3 illustrates an application for field inspection.

FIG. 4 illustrates a DNA test report of the results for a seed sample.

FIG. 5A-F are an inspection report for a processing facility.

### DETAILED DESCRIPTION

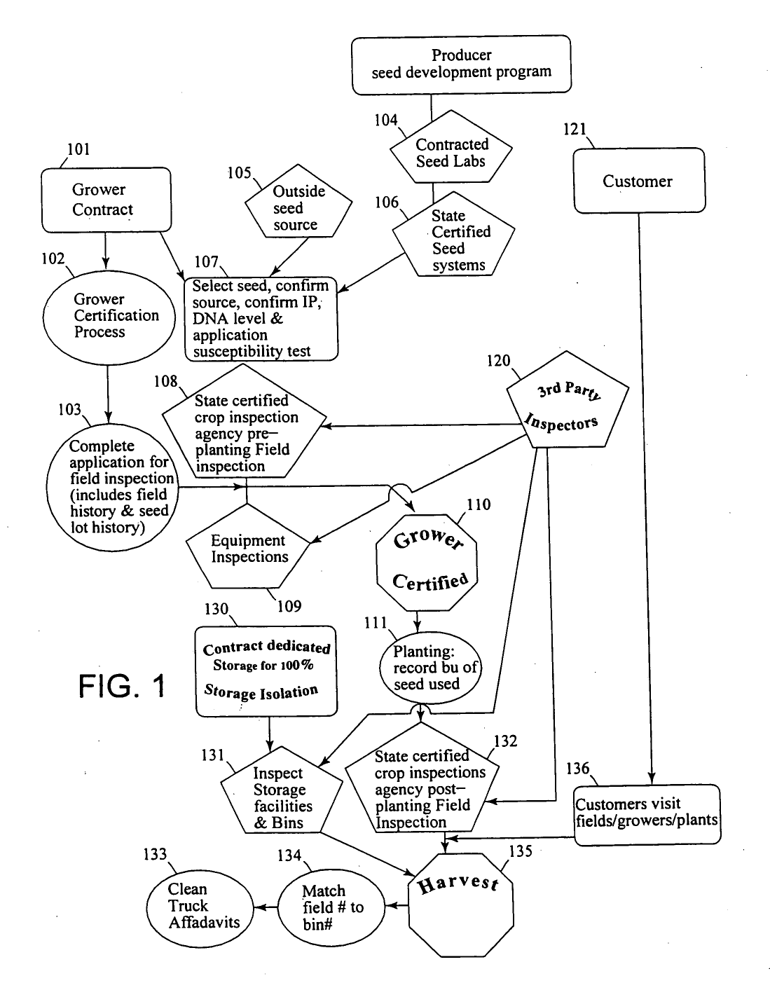

In general terms, the present invention relates to a method of creating and preserving the identity of non-genetically modified processed grain and processed food products made therefrom, utilizing testing, certification, and/or documentation throughout the entire growing production and distribution process. These procedures can begin prior to planting, proceed through the growing and harvest of grains and seeds, and can continue throughout the processing, packaging, and distribution of the grains and seeds. FIG. 1 illustrates a process flow chart for a method of preparing non-genetically modified processed grain and processed products according to one embodiment from planting through crop harvest.

One or more potential growers of a non-GMO crop can enter into a contract 101 with a seed and grain producer to plant, grow, and harvest a non-GMO crop. A seed and grain producer can be a seed supplier (e.g., a seed company), a grain broker or trader, an independent certification agency or testing laboratory, or a farm cooperative. A seed and grain producer can also be a grain or crop processor that engages in contract growing or delivery of crops to be used in one or more processing facilities, either a facility owned by the processor or a facility owned by another entity. In some embodiments, a grain or crop processor can engage in contract delivery of one or more processed food products or ingredients to be subjected to further processing.

A producer can perform various tasks directly, e.g., certification tasks such as inspecting and testing, or tasks such as growing and harvesting the crop, or tasks such as processing the harvested crop to make processed grain or a processed food product.

Alternatively, a producer can perform various tasks indirectly, e.g., a producer can enter into a contract with a grower to plant, grow and harvest a non-GMO crop with little or no contamination by GMO seeds. A producer can engage a third party to inspect a grower's land and equipment. A producer can utilize the services of an independent testing laboratory. A producer can engage a processing facility to process the harvested crop. Alternatively, a producer can accept an affidavit (i.e., an oath or declaration) from a grower, or other third party with respect to various tasks involved in the methodology. In any event, the producer accepts ultimate responsibility for certification as to the level of GMO contamination in the processed grain.

The present method is applicable to a number of different crop species. For example, non-genetically modified varieties of small grain crops such as wheat, rice, amaranth, rape, millet, and sorghum can be used. Also useful are non-genetically modified varieties of large seeded grain crops such as corn, soybeans, dry beans, kidney beans, lima beans, or coffee beans. Vegetable crop varieties that can be used in the method include non-genetically modified varieties of broccoli, peas, tomatoes, green beans, lettuce, squash, sweet corn or asparagus. Root crop varieties that are suitable for use in the method include non-genetically modified varieties of potatoes, tapioca, arrowroot, yams, sugar beets, beets, turnips, sweet potatoes, radishes, or carrots. Fruit crop varieties that are suitable for use in the method include non-genetically modified varieties of peaches, apples, pears, grapes (both table and wine), nectarines, strawberries, oranges, lemons, grapefruit, bananas, or kiwi fruit. Other crops can also be used in the method, such as herb or spice crops, and oilseed crops such as cotton.

The producer can, either directly or indirectly via a third party such as a grower, carry out grower certification and verification process 102. A certification 102 can include identification and selection of acreage suitable for growing a crop with little or no contamination by genetically modified crop material. The amount of identified acreage can be about 2 hectares, about 10 hectares or more, about 1,000 hectares or more, or about 10,000 hectares to about 100,000 hectares, or about 100,000 hectares to about 2,000,000 hectares and may involve one grower or a plurality of growers, e.g., about 2 growers, about 20 growers, about 100 growers, about 1,000 growers, about 10,000 growers, or about 100,000 growers.

As part of certification step 102, the grower or producer can document the nature of the previous years' crop for the identified acreage as well as the nature of the crops harvested on neighboring fields. Such documentation can assist in verifying that sufficient isolation distances exist for the identified acreage from fields that have grown GMO crops in the past or are growing GMO crops in the present growing season. Certification may involve, but is not limited to, determining the plant variety grown the previous season on the identified acreage, likelihood of cross-pollination from neighboring fields, and the ability to have buffer strips between the identified acreage and neighboring fields. Potential for contamination can be minimized or eliminated through, e.g. visual inspection, crop and soil tests, and the use of buffer strips for the prevention of cross-pollination from neighboring fields. Affidavits from the grower using the identified acreage or from growers using neighboring fields may also be acceptable. If it is not possible to satisfactorily minimize potential contamination on the identified acreage, the identified acreage is not used to grow a crop for the present method.

A grower or the producer may also complete an application for field inspection 103. This application typically documents the history of each field that is a part of the identified acreage, including maps, and the history of crops grown on such fields for the past one or more years. A grower may also provide documentation of seed source, equipment that has been and will be used on the fields, and current and previous crops stored on storage facilities on the farm.

Crop segregation distances, i.e., the distance between the identified acreage and fields used or to be used for growing a GMO crop of the same species, can be established and verified by designated field inspectors based on the average pollen travel distance for a particular crop species. Isolation distances between GMO and non-GMO crops are determined, in general, by using twice the generally accepted distance of travel for 98 percent of the pollen for a particular crop species. For example, if 98 percent of the pollen from a particular species travels 600 feet then the required separation distance between identified acreage and a field used for a GMO crop should be 1200 feet.

In addition, if non-GMO crops are to be planted on land that has been used to grow any other GMO crop in a previous year, the crop rotation cycle and appropriate time span between crop species must be considered before the land can be selected for use in the method. This time buffer period is determined based on the crop species to be planted. For example, a two-year delay between GMO and non-GMO soybeans is desired. The desired time buffer period for canola is three years. These time buffer periods minimize the possibility that carryover GMO seed from a previous crop season will contaminate the non-GMO crop to be planted in the present season.

Other organisms are sometimes utilized on farm land, e.g., microbes such as bacteria and fungi are sometimes used as soil additions to promote soil breakdown, fertilization or improve nutrient uptake of crops. Such organisms may be applied (e.g., as a foliar spray or a seed coating) in a non-living form or as living organisms. Land that has had an application of such genetically modified organisms in the non-living form typically is not suitable for use with non-GMO crops in the method for a period of time. For example, a time buffer period of one growing season or more may be necessary. In some embodiments, a time buffer period of one year, two years, three years, or four years or more may be necessary. Land that has had an application of such genetically modified organisms in the living form typically is not suitable for use with non-GMO crops in the method unless it can be demonstrated that the organisms are no longer present in the soil.

A producer, directly, or indirectly via a grower, also uses seed 107 for planting that is a non-GMO variety suitable for the land and growing conditions of the identified acreage. A grower or the producer verifies that a seed variety proposed for planting is a non-GMO variety. The variety can be one developed by a private company 105, a University Seed Breeding Program 104, Private Laboratory, or similar seed source. In many instances, the variety has been registered or certified through a state certification agency 106. Characteristics that can be used to confirm the non-GMO status of the variety include plant maturity, flower color, and leaf shape, although any variety-specific characteristic can be used.

Certification of little or no contamination by genetically modified plants or seeds in the plant stock or seed stock of a non-GMO variety to be planted may involve performing a genetic, biochemical, or phenotypic analysis, e.g., a DNA level and/or application susceptibility test 107. Genetic tests include DNA-type tests such as restriction fragment length polymorphism (RFLP) marker analysis, simple sequence repeat (SSR) marker analysis, isozyme analysis, nucleotide sequencing of introduced transgenes, hybridization using transgene-specific probes and polymerase chain reaction (PCR) amplification of transgene-specific sequences. An illustrative example of a genetic test for genetically modified seeds is an analysis for the presence of the cauliflower mosaic virus 35S promoter, which often is used to drive expression of a transgene coding sequence. Application susceptibility tests include phenotypic tests for the absence of a particular transgenic trait (e.g., germinating a sample of seeds and spraying the resulting seedlings to demonstrate the absence of a transgenic herbicide tolerance gene), and biochemical tests such as immunological assays and enzymatic assays. Immunological assays include enzyme-linked immunosorbent assay (ELISA) tests, in both competitive and direct formats. Various direct-binding assays may be employed in which receptors such as antibodies or binding proteins are immobilized on a solid phase. The immobilized receptor complexes are contacted with a sample to be tested which may contain an analyte of interest, e.g., a protein encoded by a transgene. Following binding of the analyte by the immobilized receptor, the solid phase is washed and then contacted with an indicator label. Competitive assay formats in which a solid phase, containing immobilized receptor or other molecule with specificity for a selected analyte, is contacted with a sample presumably containing such an analyte and with a specific competitive reagent. The specific competitive reagent comprises an analogue of the analyte and a polypeptide functioning as a label. In this embodiment, the analogue competes with the sample analyte for binding to the receptor immobilized on the solid phase. In alternative embodiments, analyte may be coupled to a solid phase and contacted with a sample and with a specific competitive reagent, that functions as a specific receptor for the analyte and as a label. In this format, sample analyte competes with solid phase analyte for binding of the soluble receptor. Radial partition immunoassay is an assay procedure in which all steps are conducted directly on a solid phase, and is also suitable for use. An illustrative example of an immunoassay for genetically modified seeds is a test that detects the presence of EPSP synthase, which can confer tolerance to the herbicide glyphosate.

Enzymatic assays can identify the presence of a transgene via an enzymatic activity expected of the protein encoded by the transgene. Such assays often measure the appearance of a fluorescent, calorimetric, chemiluminescent, phosphorescent, radiometric or hybridized product after contact with a substrate utilized the protein encoded by the transgene.

These tests confirm that the actual supply of the selected seed variety to be planted was properly grown and conditioned to maintain little or no GMO seed contamination. In some embodiments, the producer may inspect a growers' records to verify where the seeds to be planted were obtained; how much seed was purchased; how many pounds per acre the variety requires for planting; how many acres were or are to be planted; and expected final yield per acre.

Independent laboratories may be used to carry out testing procedures. Alternatively, a producer may use an in-house laboratory to carry out testing procedures. In some embodiments, a duplicate sample is sent to a plurality of laboratories in order to cross-check test results. In some embodiments, blind-tests are carried out, i.e., samples of non-GMO or GMO planting seeds, harvested crops, processed grain, or processed food products that have been purposefully contaminated are submitted to a laboratory in order to evaluate the quality of the results from that laboratory.

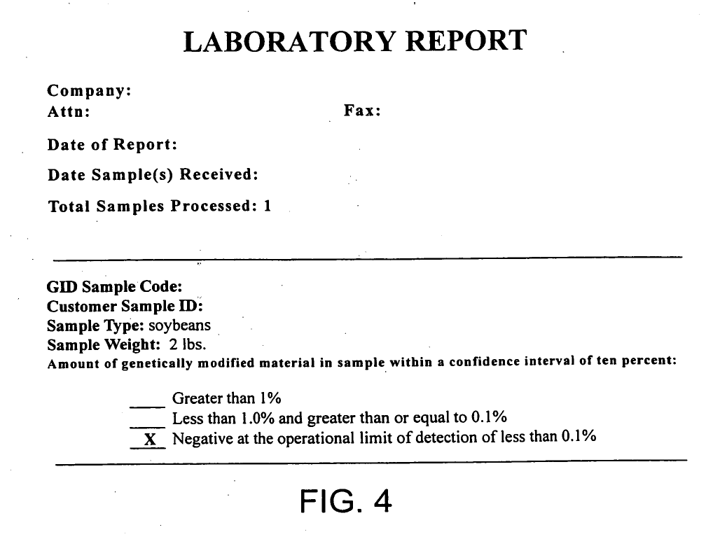

For processed grains and processed products which are labeled using language such as “ . . . may contain GMO products,” DNA level test results in the 1-5% range may be acceptable. Results from other genetic, phenotypic, or biochemical tests indicating the same range of contamination may also be acceptable. For processed grains and processed products labeled as “free of GMO products,” a more stringent standard of 0.01-0.1% contamination may be required. These more stringent standards may present a technical challenge to the current testing accuracy of DNA testing laboratories or other testing laboratories, but in combination with the verification procedures described herein, the producer can certify that contamination is, e.g., 5% or less, 1% or less, 0.1% or less, 0.01% or less, or 0.001% or less. It is also envisioned that the desired maximum GMO contamination level may be influenced by statutes and/or regulations governing various labels used on processed grain and processed food products. Because the maximum level of GMO contamination in processed grains that permits the use of a non-GMO label may vary by country, the testing performed on a particular lot of processed grain or processed food product may be modified for the country in which the lot is to be sold. FIG. 4 illustrates a DNA test report for a seed sample.

Once the planting seed is selected, the method may include a pre-planting field inspection 108 and/or corresponding equipment inspection 109. These inspections of the field to be planted and the farm equipment to be used to plant and grow the crop can be performed by the producer or by a qualified third-party inspection agency 120. A qualified inspection agency is an independent organization having the required expertise to determine plant varietal purity. A qualified inspection agency understands where potential GMO contamination could occur during planting and growing of a crop. For example, certain crop species may be more susceptible to contamination by a neighboring GMO field crop variety due to cross-pollination, whereas other crop species are less susceptible.

According to an alternate embodiment of the present invention, the grower or producer may also contact United States Department of Agriculture approved and/or International Federation of Organic Agriculture Movements (IFOAM) approved organic certifiers to inspect and certify the planting, growing, and processing of a crop. Such an activity would permit the grower to grow and harvest a crop that satisfies all applicable regulatory standards with respect to “organic” foods and also to receive certification with respect to the level of GMO contamination.

The trained inspector can also verify that equipment to be used on the identified acreage is properly maintained to minimize possible GMO contamination. Such inspections 109 may occur pre-planting, post-planting and/or pre-harvest. If a grower owns his or her own equipment, the inspector determines if it is used on GMO fields. If the equipment is used on GMO fields, the inspector verifies that proper clean-down steps are taken to minimize chances of contamination. Proper clean-down steps may include, but are not limited to, visual examination, sweeping or blowing out with air, or flushing by harvesting a pass and discarding that portion of the crop. If a grower uses a contract service for one or more planting, cultivating, fertilizing or harvesting activities, the inspector also verifies that the equipment used by these services is properly cleaned down as well. Equipment leased, rented, or borrowed by a grower which may be used on identified acreage can also be inspected as described above. Inspection of the trucks or trailers used to haul harvested crop from the fields to storage bins or processing facility is also often carried out at this time.

If a grower passes all of the pre-planting inspection steps, the grower begins to plant and grow a crop for a given season 110. The grower may record the number of bushels of non-GMO seeds used as part of planting step 111. During the growing season, the trained inspector 120 may again perform inspections of the fields and equipment 132. At the appropriate time, the crop is harvested 135.

A trained inspector can also carry out a post-planting inspection of the growing non-GMO plants to verify varietal purity. Typically, a minimum of 100 plants are examined. Off-types or variants from the morphological and physiological characteristics of the planted variety should be 0.1% or less. Any problem areas or fields can be rejected or destroyed prior to harvest. This rejection or destruction can prevent contamination of the entire crop before harvest. As an alternative, off-type or variant plants can be destroyed individually (rogueing) The trained inspector typically also makes a yield estimate per field in order to assist in gauging the final quantities of harvested crop to be expected. FIG. 3 illustrates an application for inspection of a field. The post-planting inspection also involves looking for GMO plants.

While the crop is growing, the producer may enter into contracts with customers for the ultimate delivery of non-GMO seeds and grains 121. Should such a contract be executed, the customer may wish to visit and inspect the fields, growers, and processing facilities that are or will be used to produce the processed grain 136. During these visits, the inspection results from the pre-planting inspections 108 and the pre-harvest inspections 132 may be viewed as well.

Prior to harvest, inspection 131 of storage bins and related facilities may be carried out. The producer or grower may arrange for dedicated storage 130 for the non-GMO harvested crop, e.g., in a grain elevator grain bin, a warehouse, or a refrigerated storage facility. Complete storage isolation from GMO grain should be maintained. Third party verification prior to usage that the storage bins are dedicated, segregated, contracted, and properly clean and free of GMO product is recommended. The harvested crop can be tracked by documenting quantities of non-GMO crop in and out of storage bins by establishing a lot number identification system 134. The lot number identification system identifies lots of non-GMO crop based on the field where the crop was grown, or the name of the grower. Quantities harvested from a given field, storage locations for a crop and the date when the crop was moved from a storage bin to a processing plant can also be entered into a database along with the lot number. The tracking process can include recording the test code from the processing plant container report on the invoice from processing plant. Lot number identification can also be established prior to or at planting of identified acreage, or while a crop is growing.

A single inspection of a grower may instead be performed in an alternate embodiment. In this alternate embodiment, a grower provides a producer with information which informs the producer that the particular fields being proposed for use by the grower satisfy pre-planting criteria (i.e. prior years crop, proper isolation and buffer strips etc.). If desired, a grower may provide information in the form of an affidavit. A third party inspector or the producer can inspect the fields during the growing season, and verify that the information and documentation provided by the grower are correct. The third party inspector or the producer can also verify grower compliance with post-planting/pre-harvest inspections 131 and 132. If either of these inspections reveal unsatisfactory results, the crop from that particular field is rejected.

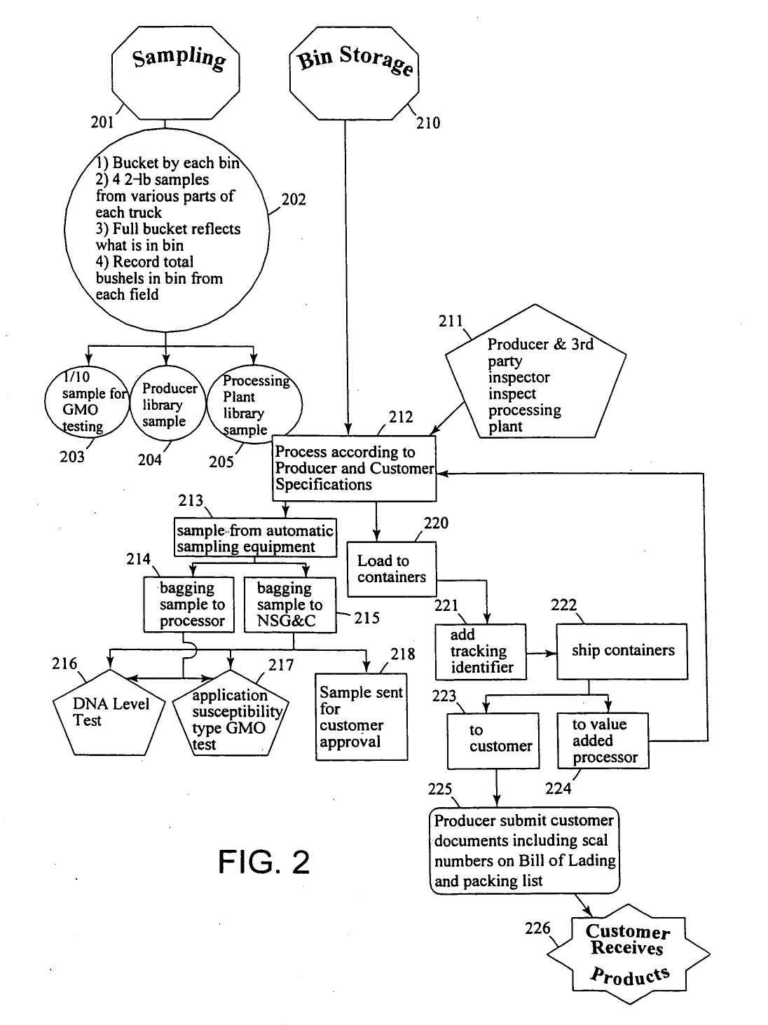

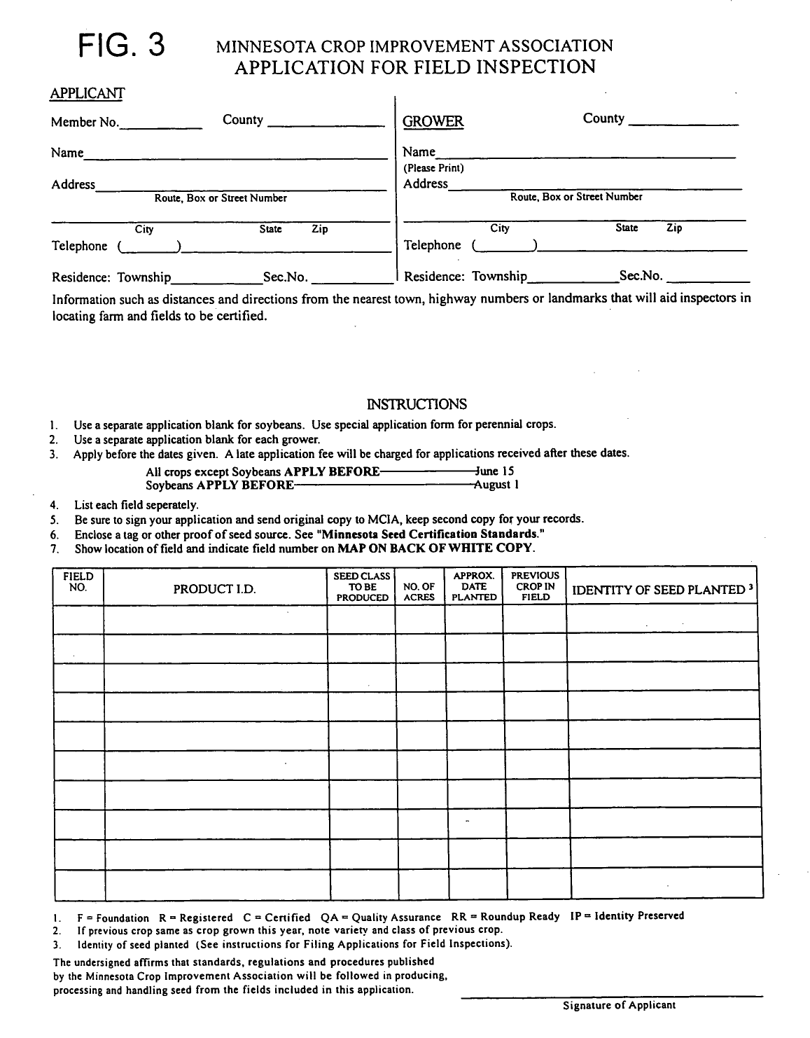

FIG. 2 illustrates a flow chart for preparing non-genetically modified processed grain, and processed food products, from crop harvest through delivery of products to customers. While the flow chart refers to a processing facility that receives a small grain or large seeded grain crop that has just been harvested, a similar flow chart is applicable to other processing facilities. For example, non-GMO soybeans may be processed initially to produce clean and bagged soybeans according to the present invention at an initial processing plant. Subsequently, another processing facility may process these soybeans into soy meal or soy oil without deviating from the spirit and scope of the present invention. In such a case, lot number identification information can be included with the processed grain as the grain or processed material moves from one processing facility to the next. Each processing facility preferably satisfies the cleaning and storage criteria necessary to minimize or prevent contamination.

A processing facility can be a facility that accepts small grain crops or large seeded crops to produce processed grain. In some embodiments, a processing facility accepts and processes vegetable crop harvests or root crop harvests, to yield fresh produce. In some embodiments, a processing facility processes a crop to yield a processed food product.

Processed food products include materials derived from a harvested crop, such as crude oils, refined, bleached and deodorized oils (e.g., soybean oil, safflower oil, sunflower oil, corn oil and canola oil), starches such as corn starch, rice starch, potato starch and wheat starch, high protein meals such as corn gluten meal and soy meal, flours such as wheat flour, corn flour or potato flour, food ingredients such as soy lecithin, carotenoids and vitamins, and corn sweeteners such as corn syrup (e.g., high fructose corn syrup) and corn maltodextrins. A grain or vegetable crop harvest can be processed by various freezing techniques to prepare processed food products such as frozen broccoli, frozen peas, frozen green beans, frozen sweet corn and the like. A grain or vegetable crop harvest can be processed using heating techniques such as roasting, pasteurizing, steaming, boiling, frying, baking or microwave treatment to prepare processed food products such as canned goods (e.g., tomato-based sauces), roasted coffee, bottled goods (e.g., catsup) and the like.

In some embodiments, material extracted from a non-genetically modified crop is combined with other food ingredients to make a value-added processed food product. For example, flour from non-genetically modified wheat can be combined with food ingredients to make breads, cakes, and the like. Soy lecithin from non-genetically modified soybeans can be combined with other food ingredients to make snacks and candy, ready-to-eat cereals, cake mixes, ice cream, margarine, baby food, mayonnaise, salad dressings, or puddings.

Samples of the harvested crop 201 from each truckload from a field can be taken 202 and used for immediate testing 203 and/or for library sample purposes 204, 205. Four two-pound portions typically are taken from various parts of the truckload for small grain crops and large seeded crops. To secure a representative sample, equal portions should be taken from evenly distributed parts of the quantity of seed to be sampled. Each portion of the sample should be visually examined for uniformity. If there appears to be a lack of uniformity, the portions should each be tested separately. If the portions are uniform, they can be combined to form a composite sample. Clean truck affidavits 133 may be used for trucks, rail cars or other transport carriers that transport grain from field to storage bin 210, and from storage bin 210 to processing plant. Clean truck affidavits verify that trucks are properly maintained and that proper procedures to minimize contamination were carried out. In the event that any contamination is found, the corresponding portion of the grain is rejected. Each storage bin is numbered and all incoming and outgoing activities records can be submitted for inspection.

Additional samples may be taken at the processing facility. Sampling procedures that are appropriate for the crop to be processed and for the type of processed food product to be made should be employed. As the harvested crop is processed within a processing plant, samples can be generated using automatic sampling equipment 213. Typically, some type of continuous flow automatic sampling device is recommended in order to collect a plurality of samples. This device should take a small sample every few minutes during processing. This sampled product can be kept on a batch lot basis and used for testing of each lot. Samples can be used immediately for testing or saved for testing at a later date by a processor 214 or a producer 215. Suitable tests include a DNA level test 216, application susceptibility type GMO test 217, or other types of tests as discussed above. A portion of such samples can also be maintained within a library 204 in order to permit later testing. A portion of such samples may also be sent to a customer for approval, as desired 218. Results of tests of processed food products provide an estimate of the amount of genetically modified crop material that has contaminated the processed food product.

Before samples are sent to the laboratory for analysis, the containers should be properly identified with variety, seed class, grower's name, tests to be performed and lot number. These additional samples are submitted to different laboratories or tested internally using, e.g., application susceptibility tests and/or DNA level testing. If contamination is found, a second verifying test is performed. If the second test confirms contamination, that lot of processed grain is rejected. Samples can be taken if desired and sub-divided into portions for testing, processing plant library sample, producer library sample, and customer review and approval.

Phenotypic, biochemical or genetic test results can also be used for value-added processed food products to estimate the amount of GMO contamination in the value-added product. Such an estimate is based on the percent GMO contamination of each ingredient in a value-added food product and the proportion of that ingredient present in the value-added product.

The level of purity desired in processed grain, processed food product, or value-added food product, can be adjusted according to food labeling requirements set by governmental statutes or regulations, e.g., about 0.001% contamination, about 0.01% contamination, about 0.02% contamination, about 0.03% contamination, about 0.04% contamination, about 0.05% contamination, about 0.1% contamination, about 0.3% contamination, about 0.7% contamination, about 1% contamination, about 2% contamination, or about 5% contamination. To meet various levels of purity, the disclosed method can be adjusted by accepting the desired contamination level from genetic, phenotypic or biochemical tests, by accepting the desired contamination level during inspections, or by accepting the desired contamination level in equipment and machinery.

In some embodiments, the harvested and tested crop is taken from bin storage 210 and processed in a processing facility according to producer or customer specifications 212 to generate the deliverable product. Prior to processing of the crop, the processing plant can be inspected 211 for compliance with the requirements of the .present invention. This inspection can be performed by a qualified third party inspection agency such as a governmental seed and crop inspection agency. Alternatively, an inspection can be performed by the producer or an affidavit from the processing facility can be accepted. Such an inspection can include, but is not limited, to annual and spot inspections. In general, only processing plants that meet industry-specific quality standards for quality control should be used.

An example of a suitable facility for processing small grain and large seeded grain crops is a processing facility that meets or exceeds the requirements to be a certified seed conditioner. The criteria utilized for such crops typically include a set of general requirements, a set of machinery and equipment requirements, and a set of other storage related requirements. All three sets of requirements typically are satisfied before the processing plant is considered suitable to accept non-GMO small grain or large-seeded grain crops.

The general requirements include documentation showing compliance with applicable state and federal seed laws and certification requirements. Typically, a processing plant is inspected at least once a year to ensure compliance with such laws and regulations.

The machinery and equipment requirement typically includes the use of an air-screen mill equipped with mechanical screen cleaners for cleaning the non-GMO crop. The processing plant should possess on-site weighing and testing equipment necessary to meet its needs. Bag closing equipment should be present on-site if bagged seeds are handled. Compressed air or commercial vacuum cleaning systems typically are present in the processing plant to clean equipment, plant facilities, and storage containers between the processing of all lots.

The remaining requirements relate to requirements concerning pits, bins, legs, heads and distributors. All elevator pits preferably are cleanable. It is preferable that all pits possess a metal lining, and that pits possess a securely fitting cover. Additionally, the valley angle within all pits should be at least 45 degrees. All bins at the processing plant should be tightly constructed and easily cleanable. Hopper buttons should be made of metal or metal lined. All bins at the processing plant should possess a unique identification number to permit tracking the processing of a given lot of non-GMO crop.

A screen mill desirably possesses at least two legs: one for intake of unprocessed crop and one for outgoing cleaned seed. A third leg is, however, recommended for screening only. Elevator boots should be raised at least eight inches above the floor to permit them to be readily opened for cleaning. Plastic cups are optional for elevator legs. All distributors should possess an inspection door to permit easy cleaning. All spouting should be constructed of heavy gauge material and steeply angled. Flex spouting typically is not used for processing non-GMO crops, unless it can be certified that the processing plant only accepts non-GMO crops. Rivets and bolts should not protrude into the spouts.

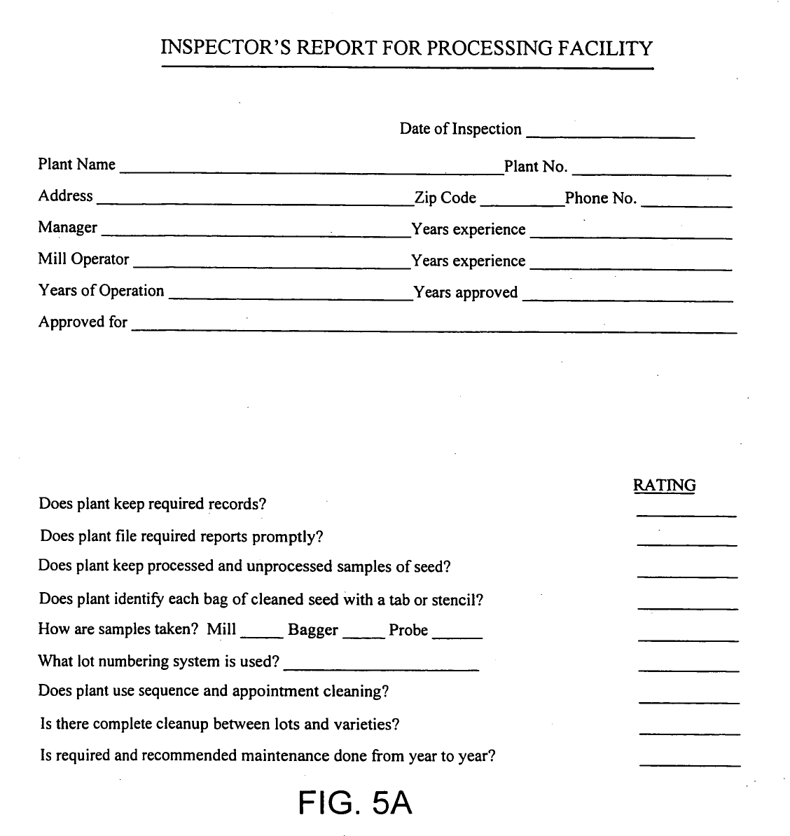

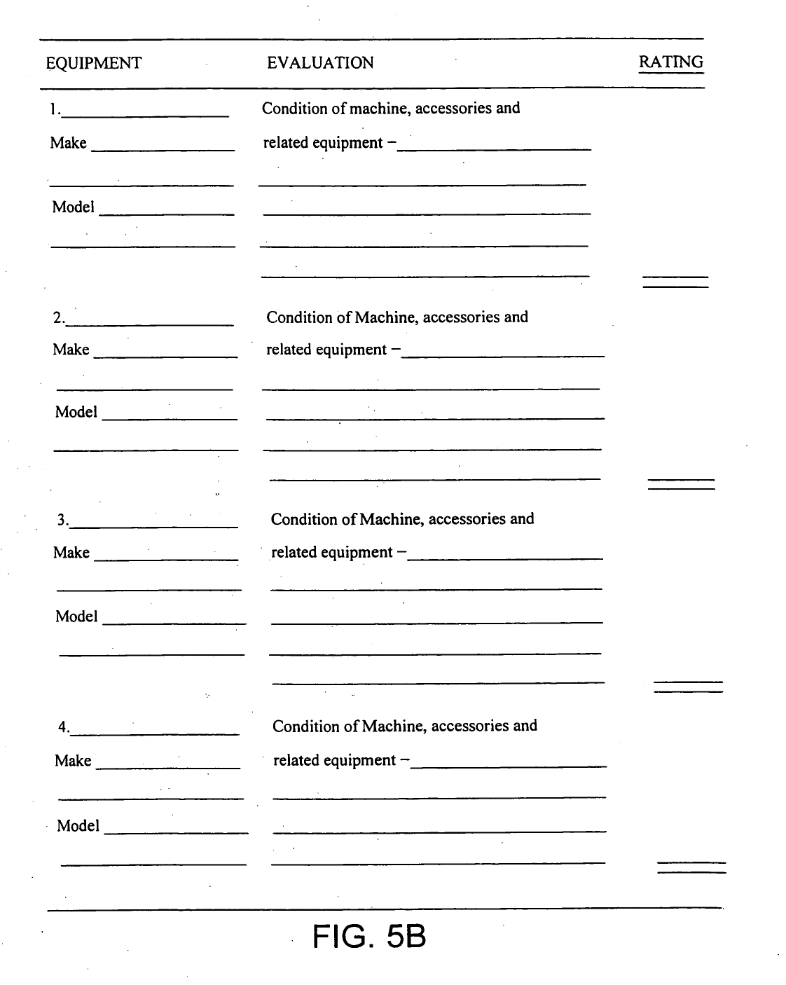

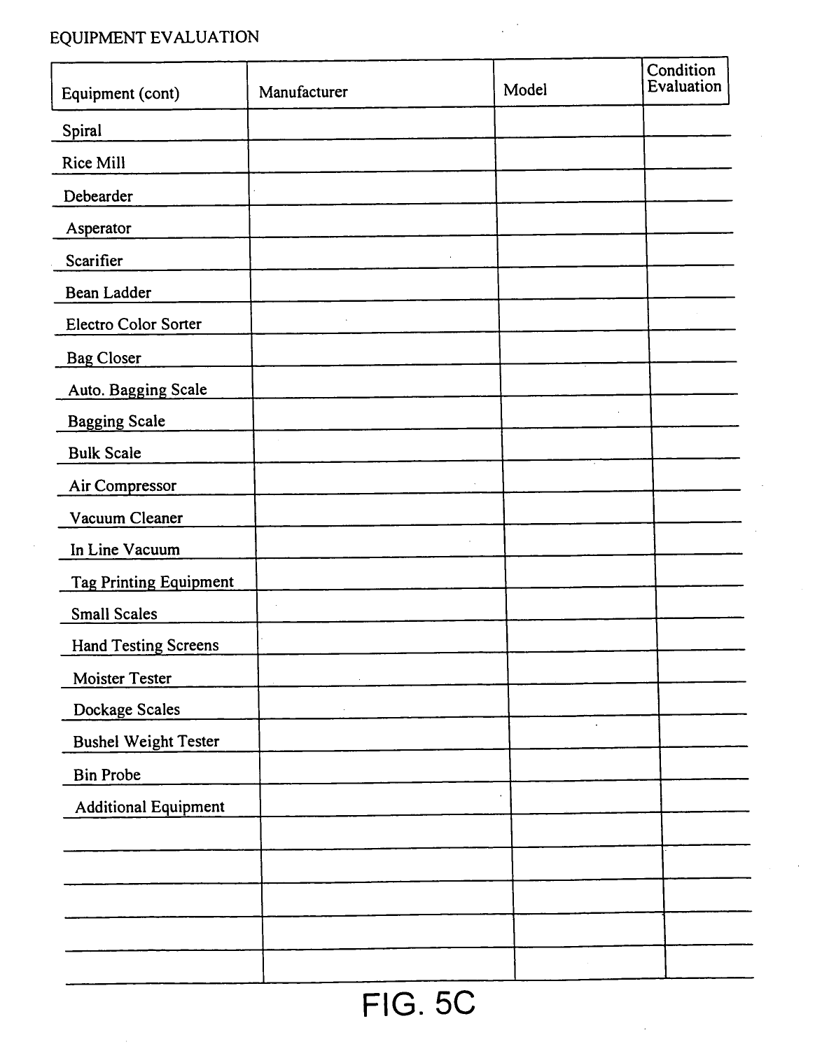

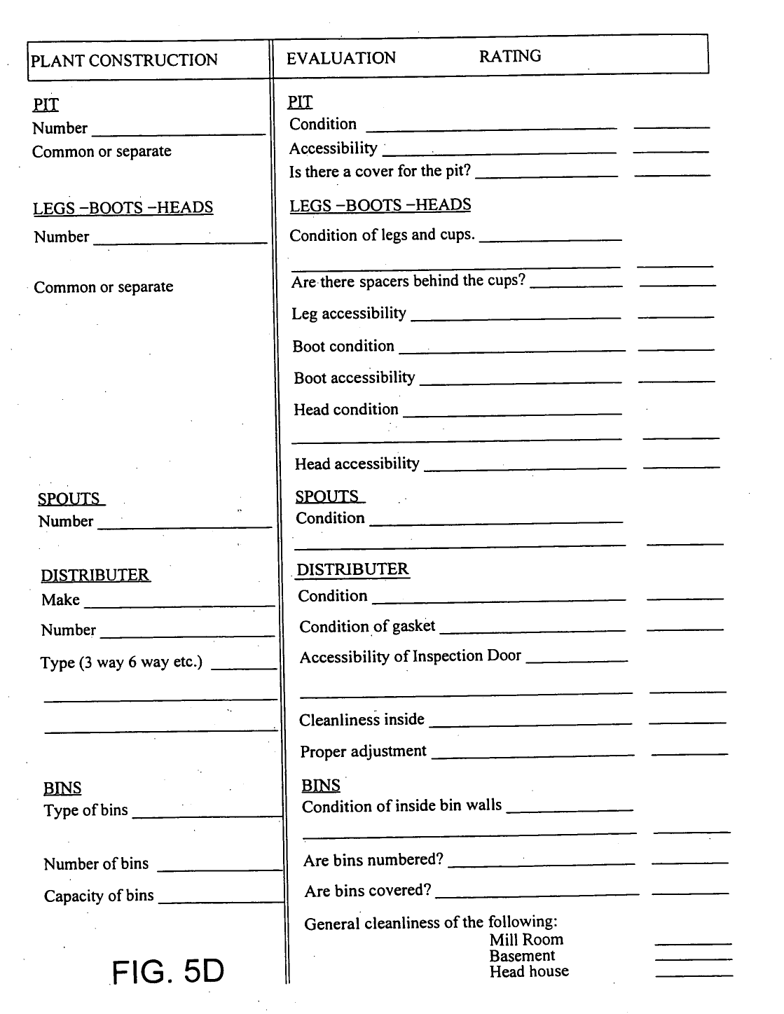

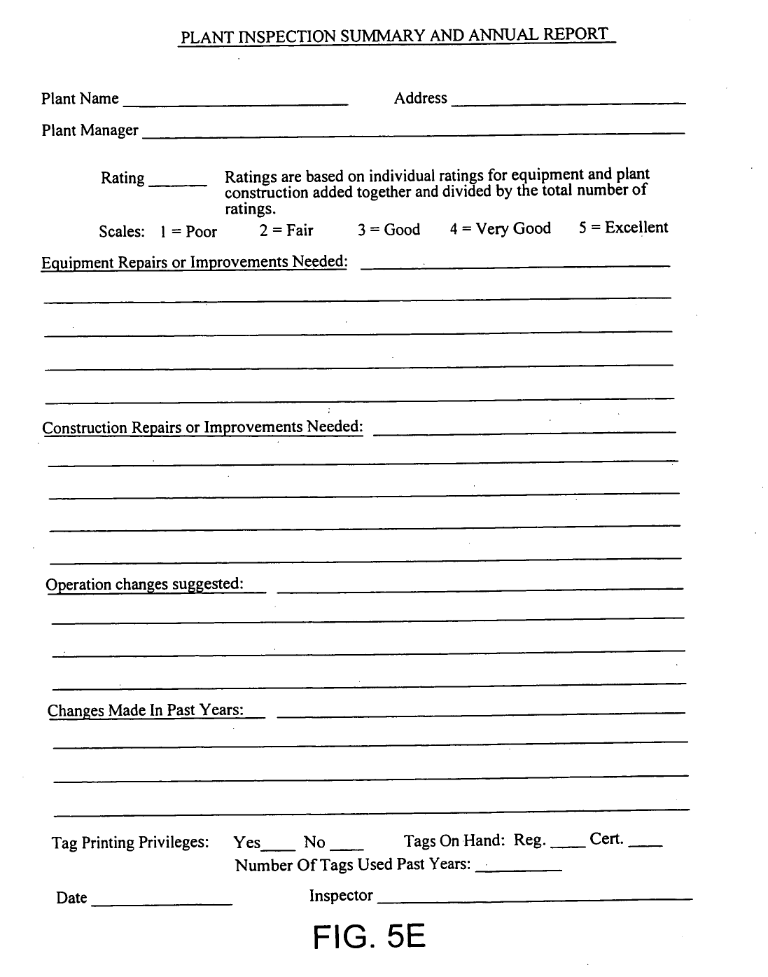

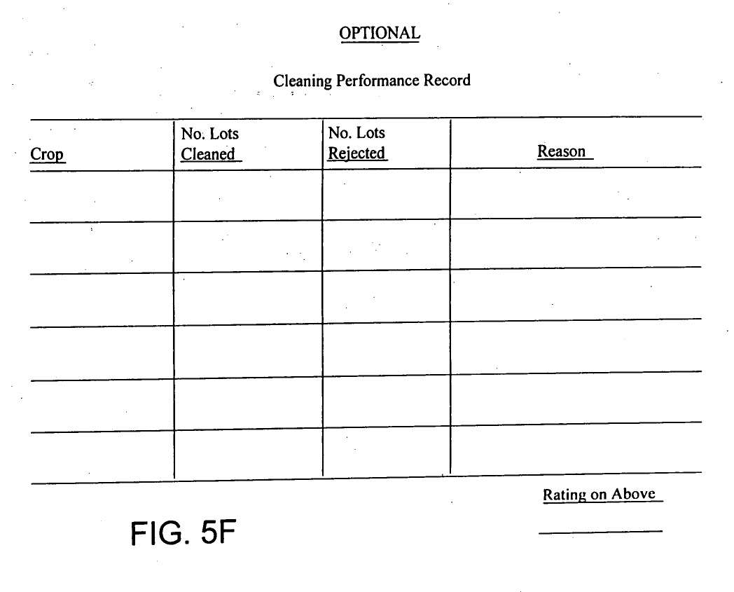

Satisfaction of industry-specific quality standards assures the producer and customer that the processing facility possesses the ability to keep lots of harvested crops appropriately segregated, to implement proper clean-down procedures, and to properly track lots being processed. FIG. 5A-F shows an inspection report that may be used by an inspection and quality designator to verify that a processing plant for small grain and large-seeded grain crops possesses such abilities.

A processing facility typically has the ability to pack non-GMO processed grain or food products in several manners, such as bags, totes, bulk to ocean containers, rail cars, or trucks 220. Each package of non-GMO material can possess lot identification numbers discussed above which contain information concerning the source and identity of the processed material in the package. In the case of bulk containers, the lot ID number information may include a seal affixed to the container holding the non-GMO product where the seal references the container number. In the case of paper bags or totes, lip-printing, sewn tags or similar materials can be used to affix lot identification number information to the package to facilitate tracking of product 221. From this lot identifier information, the field where the product was grown, the variety, storage, and related handling information can be deduced in some embodiments. Packaging materials used to enclose non-GMO processed grain are typically those used for food purposes and typically are able to minimize or prevent contamination of the enclosed processed grain or food product by GMO organisms. Packaged product can be delivered 222 to a value-added processor 224, or directly to a customer 223 if the product is in its final level of processing for the particular customer in question. The product can be shipped with seal numbers on a Bill of Lading and packing list 225, for customer convenience 226. Other documentation for a customer may include lot codes, test results, ingredient composition, nutritional labels, quality certificates, phytosanitary certificates, and the like.

In some embodiments, methods of the invention can be used to prepare genetically modified processed grain or processed food products and thereby minimize or prevent contamination of such grain by non-genetically modified seeds or crop material. Such methods can comprise selecting genetically modified seeds for planting; certifying that the genetically modified seeds were planted and grown under conditions effective for harvesting a crop containing 5% or less non-genetically modified seeds; harvesting the crop; processing the crop under conditions effective for producing processed grain containing 5% or less non-genetically modified seeds; and certifying that the harvested crop was processed under those processing conditions.

Methods of the invention are useful for genetically modified crop varieties that either produce valuable nutrients or pharmaceuticals that are not normally present in that crop, or that produce elevated levels of nutrients or pharmaceuticals which are normally present. Suitable varieties include varieties of all those species described above, e.g., corn, soybean, rice, wheat, rape, sorghum, millet, amaranth, beans (including kidney beans, lima beans, dry beans, green beans), coffee beans and alfalfa.

A crop harvested from a genetically modified variety, containing little or no contamination by non-genetically modified crop material or contamination by crop material from genetically modified varieties containing unwanted genetic modifications, can be subjected to processing as described herein in order to make various nutrients and/or pharmaceuticals. Suitable nutrients or pharmaceuticals include antigenic molecules for oral vaccination, vitamins (e.g., vitamin A and vitamin E), food ingredients (e.g., lecithins, dextrins, amylodextrins, pectins, saccharides, proteins and peptides, and antioxidants), cytokines, anticancer agents, human growth hormones, inhibitors of seed toxins (e.g., inhibitors of soybean trypsin inhibitor), and the like. For example, the disclosed methods can be used with a rice variety that has been genetically modified to produce vitamin A, to yield processed grain that has little or no contamination by non-genetically modified rice or genetically modified rice having an unwanted genetic modification such as herbicide.

Processed grain, processed food products, nutrients and/or pharmaceuticals from a genetically modified variety can be produced by the disclosed methods such that contamination by non-genetically modified seeds or crop material is 5% or less, 4% or less, 3% or less, 2% or less, 1% or less, 0.5% or less, 0.2% or less, 0.1% or less, 0.05% or less, or 0.01% or less. Phenotypic tests such as application susceptibility tests can be used to show that 95% or greater, 99% or greater, 99.9% or greater, 99.95% or greater, or 99.99% or greater of the planting seeds are in fact genetically modified. A corresponding application susceptibility test can be carried out during or after processing of the harvested crop. Other suitable tests that can be used include biochemical tests for the presence of the expected nutrient or pharmaceutical or genetic tests to confirm the presence of nucleic acid expected to be present in the genetically modified variety can also be used. Suitable genetic tests include, without limitation, the use of PCR, hybridization, sequencing analyses, and the like. Genetic tests can be used to show that 95% or greater, 99% or greater, 99.9% or greater, or 99.99% or greater of the planting seeds are in fact genetically modified. A corresponding genetic test can be carried out during or after processing of the harvested crop.

Certification techniques described above can be readily adapted for use in minimizing contamination of a GMO crop by non-GMO seeds or crop material, or seeds or crop material from unwanted GMO varieties.

In some embodiments, material extracted from a genetically modified crop is combined with other food ingredients to make a value-added processed food product. For example, flour from genetically modified wheat can be combined with food ingredients to make breads, cakes, and the like. Antioxidants from genetically modified soybeans can be combined with other food ingredients to make snack foods, ready-to-eat cereals, cake mixes, or puddings.

Samples of a harvested crop from a genetically modified variety 201 from each truckload from a field can be taken 202 and used for immediate testing 203 and/or for library sample purposes 204, 205, as discussed above. Additional samples may be taken at the processing facility. Sampling procedures that are appropriate to each commodity and processed food product should be employed, using automatic sampling equipment 213, as discussed above.

Phenotypic, biochemical or genetic test results can also be used for value-added processed food products or pharmaceuticals made from genetically modified crops or processed crop material in order to estimate the amount of contamination by non-genetically modified crop material in the processed food product or pharmaceutical. The level of purity desired for a pharmaceutical product may be higher than that desired for processed grain or value-added processed food products. For example, the level of purity required by government regulation or statute for a pharmaceutical obtained from a genetically modified crop may be 99.999% purity (i.e., less than 0.0001% contamination by crop material from non-genetically modified species). The level of purity required for a value-added food ingredient made from a genetically modified crop, on the other hand, may be 0.01%.

In some embodiments, a processing facility for a genetically modified crop is inspected by a qualified third party inspection agency as discussed above. Alternatively, an inspection can be performed by the producer or an affidavit from the processing facility can be accepted. Typically, documentation exists for a processing facility showing compliance with applicable state and federal laws and certification requirements. In some instances, a processing facility has documentation showing compliance with good pharmaceutical manufacturing practices. Lot number identification information can be included with the processed grain as the grain or processed material moves from one processing facility to the next. Each processing facility typically satisfies the cleaning and storage criteria necessary to minimize or prevent contamination by non-genetically modified seeds or crop material.

A processing facility typically has the ability to pack genetically modified processed grain, processed food products, nutrients and/or pharmaceuticals in a suitable manner. Each package of genetically modified processed grain, processed food product, nutrient or pharmaceutical can possess lot identification numbers as discussed above which contain information concerning the source and identity of the processed material in the package. For pharmaceuticals, a lot identification number may be necessary to meet government regulatory or statutory requirements. Machinery and equipment in a facility that processes genetically modified crops typically meets the machinery and equipment requirements discussed above for non-genetically modified grain crops. An inspection report may be used to verify that a processing plant possesses such abilities. Processing plants working with bulk grain, as well as value-added processing plants that process a crop to produce value-added food products, pharmaceuticals or nutrients, may use an inspection report as discussed above.

The foregoing description of the invention has been presented for the purposes of illustration and description. It is not intended to be exhaustive or to limit the invention to the precise form disclosed. Many modifications and variations are possible in light of the above teaching. It is intended that the scope of the invention be limited not with this detailed description, but rather by the claims appended hereto.

1. A method for preparing a non-genetically modified processed food product, comprising:
a) certifying that non-genetically modified seeds were grown under conditions effective for harvesting a crop containing 5% or less genetically modified seeds; and
b) certifying that said crop was processed under conditions effective for producing said processed food product, said food product containing 5% or less genetically modified crop material.

2-51. (canceled)
